(19) United States
(12) Patent Application Publication
(10) Pub. No.: US 2025/0160870 A1
(43) Pub. Date: May 22, 2025

(54) PERICARDIAL TRANSECTION DEVICE WITH RETRACTABLE CUTTING APPARATUS

(71) Applicant: EDWARDS LIFESCIENCES CORPORATION, Irvine, CA (US)

(72) Inventors: Daniel James Murray, Orange, CA (US); Yaeer E. Lev, Torrance, CA (US); Jose Islas, Corona, CA (US)

(21) Appl. No.: 18/954,953

(22) Filed: Nov. 21, 2024

Related U.S. Application Data
(63) Continuation of application No. PCT/US2023/023759, filed on May 26, 2023.
(60) Provisional application No. 63/347,531, filed on May 31, 2022.

(51) Int. Cl. A61B 17/32; A61B 17/3211; A61B 18/00; A61B 18/14

(52) U.S. Cl. CPC A61B 17/320016; A61B 18/1492; A61B 2017/32113; A61B 2018/00351; A61B 2018/00601; A61B 2018/1475

(57) ABSTRACT

A medical device includes an incision device operably coupled to a distal end of an elongate catheter. The incision device includes an incision channel, and a retractable cutting apparatus disposed within the incision channel. The retractable cutting apparatus includes a first cutting apparatus link that is rotatably fixed proximate to a distal end of the incision channel and a second cutting apparatus link that is attached to the first link proximal end of the first cutting apparatus link. The first cutting apparatus preferably includes a first cutting surface proximate to the first link proximal end of the first cutting apparatus link and the second cutting apparatus link includes a second cutting surface. The retractable cutting apparatus also includes a guide member coupled to cause the retractable cutting apparatus to move between a retracted position and a deployed position.

### RELATED APPLICATIONS

This application is a continuation application of International Patent Application No. PCT/US2023/023759, filed May 26, 2023, which claims the benefit of U.S. Provisional Application No. 63/347,531, filed on May 31, 2022. Each of the foregoing patent applications are hereby incorporated by reference in their entireties.

### TECHNICAL FIELD

This disclosure is directed to devices and methods for cutting in the pericardium. The devices and methods are generally applicable to the treatment of heart failure, for example, heart failure with preserved ejection fraction (HFpEF) or reduced ejection fraction (HFrEF) by introducing one or more incision lengths in a pericardium, e.g., a parietal layer.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In one example, a medical device for creating elongated incisions within a pericardium is provided. The medical device includes a catheter including at least one lumen, a longitudinal axis, a proximal end, and a distal end. The medical device also includes an incision device operably coupled to the distal end of the catheter. The incision device includes an incision channel defined along the incision device and the incision channel defines an open channel top and a channel bottom. The medical device further includes a retractable cutting apparatus disposed within the incision channel. The retractable cutting apparatus includes a first cutting apparatus link defining a first link distal end and a first link proximal end. The first link distal end is rotatably fixed proximate to a distal end of the incision channel. The retractable cutting apparatus also includes a second cutting apparatus link defining a second link distal end and a second link proximal end, wherein the second link distal end is attached to the first link proximal end of the first cutting apparatus link. The retractable cutting apparatus further includes a guide member coupled to the second link proximal end. The guide member is structured to move the first cutting apparatus link and the second cutting apparatus link and cause the retractable cutting apparatus to move between a retracted position and a deployed position. The retractable cutting apparatus also includes a first blade attached to the first link proximal end of the first cutting apparatus link. The first blade is structured to protrude from the incision channel in an instance in which the retractable cutting apparatus is in the deployed position. In an instance in which the guide member is moved in a first direction of the first cutting apparatus link and the second cutting apparatus link, the first link proximal end of the first cutting apparatus link and the second link distal end of the second cutting apparatus link are structured to move in a direction of the open channel top to the deployed position. In an instance in which the guide member is moved in a second direction opposite of the first cutting apparatus link and the second cutting apparatus link, the first link proximal end of the first cutting apparatus link and the second link distal end of the second cutting apparatus link are structured to move in a direction of the channel bottom to the retracted position

In some example embodiments, alone or in combination with any of the previous example embodiments, in an instance in which the first blade is in the retracted position, the first blade is disposed within the incision channel. In some example embodiments, alone or in combination with any of the previous example embodiments, in an instance in which the first blade is in the deployed position, the first blade at least partially protrudes from the incision channel. In some example embodiments, alone or in combination with any of the previous example embodiments, the cutting apparatus is structured to be moveable between the retracted position and the deployed position upon placement of the incision device within a pericardium of the human body.

In some example embodiments, alone or in combination with any of the previous example embodiments, the device includes a sheath structured to cover the incision channel in an instance in which the retractable cutting apparatus. In some example embodiments, alone or in combination with any of the previous example embodiments, the second cutting apparatus link defines a first blade receiving aperture that is structured to receive the first blade in an instance in which the retractable cutting apparatus is in the retracted position.

In some example embodiments, alone or in combination with any of the previous example embodiments, upon movement to the deployed position, the first blade is structured to make an incision along a pericardium upon movement of the catheter.

In some example embodiments, alone or in combination with any of the previous example embodiments, at least one of the first cutting apparatus link or the second cutting apparatus link is a first blade. In some example embodiments, alone or in combination with any of the previous example embodiments, the first cutting apparatus link and the second cutting apparatus link are each a blade. In some example embodiments, alone or in combination with any of the previous example embodiments, the first cutting apparatus link and the second cutting apparatus link are attached to one another via a hypotube connection.

In some example embodiments, alone or in combination with any of the previous example embodiments, the incision channel includes a channel protrusion structured to prevent the first blade and the second blade from being parallel along the incision channel. In some example embodiments, alone or in combination with any of the previous example embodiments, the channel protrusion causes an angle between the first blade and a secondary blade to be less than 180 degrees to assist the movement between the retracted position and the deployed position.

In some example embodiments, alone or in combination with any of the previous example embodiments, the incision is linear along the pericardium. In some example embodiments, alone or in combination with any of the previous example embodiments, the incision is non-linear along the pericardium. In some example embodiments, alone or in combination with any of the previous example embodiments, the first blade remains stationary along the incision channel in an instance in which the catheter is moving along the pericardium.

In some example embodiments, alone or in combination with any of the previous example embodiments, the retractable cutting apparatus further includes a secondary blade structured to compliment the first blade during cutting. In some example embodiments, alone or in combination with any of the previous example embodiments, the first blade defines a cutting surface, and the secondary blade defines a secondary cutting surface with each of the cutting surface and the secondary cutting surface being structured to cut into the pericardium.

In some example embodiments, alone or in combination with any of the previous example embodiments, the cutting surface and the secondary cutting surface are structured to cut in opposite directions, such that a scissor effect or glide effect occurs. In some example embodiments, alone or in combination with any of the previous example embodiments, the first blade is rigidly attached to the first cutting apparatus link. In some example embodiments, alone or in combination with any of the previous example embodiments, the secondary blade is rigidly attached to the secondary cutting apparatus link.

In some example embodiments, alone or in combination with any of the previous example embodiments, the first blade defines a blade height that is defined by the amount the first blade protrudes from the incision channel in the deployed position. In some example embodiments, alone or in combination with any of the previous example embodiments, the blade height is adjustable. In some example embodiments, alone or in combination with any of the previous example embodiments, the blade height is adjustable via a lead screw.

In some example embodiments, alone or in combination with any of the previous example embodiments, each of the first cutting link, the second cutting link, and the guide member are positioned along a first plane in an instance in which the retractable cutting apparatus is in the retracted position. In some example embodiments, alone or in combination with any of the previous example embodiments, the first cutting link and the second cutting link define a v-shaped protruding from the incision channel.

In some example embodiments, alone or in combination with any of the previous example embodiments, a linkage distance is defined between the first link distal end of the first cutting link and the second link proximal end of the second cutting link with the linkage distance being reduced during the movement of the retractable cutting apparatus from the retracted position to the deployed position.

In some example embodiments, alone or in combination with any of the previous example embodiments, the catheter is steerable. In some example embodiments, alone or in combination with any of the previous example embodiments, at least a portion of the catheter is radiopaque. In some example embodiments, alone or in combination with any of the previous example embodiments, at least a portion of at least one of the first cutting apparatus link or the second cutting apparatus link is radiopaque. In some example embodiments, alone or in combination with any of the previous example embodiments, at least a portion of the first cutting apparatus link and the second cutting apparatus link are radiopaque.

In some example embodiments, alone or in combination with any of the previous example embodiments, the device includes an introducer positioned near the distal end of the catheter. In some example embodiments, alone or in combination with any of the previous example embodiments, the introducer is a puncturing tip structured to provide access to the pericardium. In some example embodiments, alone or in combination with any of the previous example embodiments, the catheter is structured to provide enough force to the introducer to puncture human tissue to allow access to the pericardium. In some example embodiments, alone or in combination with any of the previous example embodiments, at least a portion of the introducer is radiopaque.

In some example embodiments, alone or in combination with any of the previous example embodiments, the at least one lumen includes a guidewire lumen. In some example embodiments, alone or in combination with any of the previous example embodiments, the guide member is slidably positioned within the guidewire lumen. In some example embodiments, alone or in combination with any of the previous example embodiments, the first blade includes a cutting surface, and the cutting surface is positioned to cut tissue upon being moved within the human body in an instance in which the first blade is in the deployed position.

In some example embodiments, alone or in combination with any of the previous example embodiments, the cutting surface of the first blade when laterally projected, faces away from the distal end and towards the proximal end of the catheter. In some example embodiments, alone or in combination with any of the previous example embodiments, at least a portion of the cutting apparatus is radiopaque.

In some example embodiments, alone or in combination with any of the previous example embodiments, the first blade is structured to have a sufficient force to puncture pericardial tissue in an instance in which the first blade is moved from the retracted position to the deployed position.

In some example embodiments, alone or in combination with any of the previous example embodiments, the medical device is sterilized. In some example embodiments, alone or in combination with any of the previous example embodiments, the medical device can be sterilized via gamma sterilization. In some example embodiments, alone or in combination with any of the previous example embodiments, the medical device can be sterilized via ethylene oxide sterilization. In some example embodiments, alone or in combination with any of the previous example embodiments, the medical device can be sterilized via autoclave sterilization. In some example embodiments, alone or in combination with any of the previous example embodiments, the medical device can be sterilized via E-beam sterilization.

In some example embodiments, alone or in combination with any of the previous example embodiments, the first blade includes a puncture tip with the puncture tip being a sharp edge structured to piece pericardium tissue.

In some example embodiments, alone or in combination with any of the previous example embodiments, the device includes a guide member movement actuator attached to the guide member with the guide member movement actuator being manipulated to move the guide member. In some example embodiments, alone or in combination with any of the previous example embodiments, the guide member apparatus movement actuator is actuated by a catheter controller.

In some example embodiments, alone or in combination with any of the previous example embodiments, the device includes a controller structured to move the guide member. In some example embodiments, alone or in combination with any of the previous example embodiments, the controller is further structured to move the catheter. In some example embodiments, alone or in combination with any of the previous example embodiments, the controller is structured to move the sheath between a covered position and an uncovered position with the sheath covering the incision channel in the covered position.

In some example embodiments, alone or in combination with any of the previous example embodiments, the sheath has a distal end being slidably locatable on the catheter. In some example embodiments, alone or in combination with any of the previous example embodiments, the sheath further includes at least one opening adjacent the distal end of the sheath. In some example embodiments, alone or in combination with any of the previous example embodiments, the distal end of the sheath is traversable along the catheter. In some example embodiments, alone or in combination with any of the previous example embodiments, the at least one opening of the sheath is traversable along the catheter to cover the incision channel or to uncover the incision channel.

In some example embodiments, alone or in combination with any of the previous example embodiments, at least a portion of the distal end of the sheath and at least a portion of the incision channel are radiopaque to align the distal end of the sheath and the incision channel. In some example embodiments, alone or in combination with any of the previous example embodiments, the at least one opening of the sheath is traversable to align with the incision channel, allowing the first blade to laterally project from the incision channel.

In some example embodiments, alone or in combination with any of the previous example embodiments, the at least one opening of the sheath is traversable to align with the incision channel, allowing the actuator to cause the cutting surface to laterally project from the incision channel.

In some example embodiments, alone or in combination with any of the previous example embodiments, the device includes one or more stabilizing members structured to maintain the catheter at a given location within the human body. In some example embodiments, alone or in combination with any of the previous example embodiments, the one or more stabilizing members are structured to move between a stabilizing retracted position and a stabilizing deployed position. In some example embodiments, alone or in combination with any of the previous example embodiments, the one or more stabilizing members are moved from the stabilizing retracted position to the stabilizing deployed position upon positioning of the catheter within the human body.

In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures.

In some example embodiments, alone or in combination with any of the previous example embodiments, the one or more openings of the sheath are traversable along the catheter allowing an actuator to cause the one or more stabilizing members to laterally project through both the catheter and the sheath. In some example embodiments, alone or in combination with any of the previous example embodiments, the actuator concurrently or sequentially laterally projects the incision device and the one or more stabilizing members through the one or more openings of the catheter and the one or more openings of the sheath.

In some example embodiments, alone or in combination with any of the previous example embodiments, the distal end of the sheath concurrently or sequentially allows the first blade and the one or more stabilizing members to laterally project from the catheter. In some example embodiments, alone or in combination with any of the previous example embodiments, the first blade is assisted in cutting into the pericardium by a force provided by the one or more stabilizing member.

In some example embodiments, alone or in combination with any of the previous example embodiments, the retractable cutting apparatus includes an electrode. In some example embodiments, alone or in combination with any of the previous example embodiments, the retractable cutting apparatus includes at least one blade and at least one electrode. In some example embodiments, alone or in combination with any of the previous example embodiments, the electrode is structured to receive current and/or radio frequency energy to ablate, burn, vaporize, and/or separate tissue.

In some example embodiments, alone or in combination with any of the previous example embodiments, an electrical current is provided to the retractable cutting apparatus. In some example embodiments, alone or in combination with any of the previous example embodiments, at least one of the first blade or the secondary blade is an electrode. In some example embodiments, alone or in combination with any of the previous example embodiments, the first blade and the secondary blade are each an electrode.

In some example embodiments, alone or in combination with any of the previous example embodiments, the device also includes at least one nerve detection device. In some example embodiments, alone or in combination with any of the previous example embodiments, the at least one nerve detection device is located on the catheter. In some example embodiments, alone or in combination with any of the previous example embodiments, the at least one nerve detection device is located adjacent the incision device. In some example embodiments, alone or in combination with any of the previous example embodiments, the at least one nerve detection device is located on the puncturing tip. In some example embodiments, alone or in combination with any of the previous example embodiments, the at least one nerve detection device is located on the cutting surface.

In some example embodiments, alone or in combination with any of the previous example embodiments, the device includes at least one nerve stimulation device. In some example embodiments, alone or in combination with any of the previous example embodiments, the at least one nerve stimulation device is located on the catheter. In some example embodiments, alone or in combination with any of the previous example embodiments, the at least one nerve stimulation device is located adjacent the incision device. In some example embodiments, alone or in combination with any of the previous example embodiments, the at least one nerve stimulation device is located on the puncturing tip. In some example embodiments, alone or in combination with any of the previous example embodiments, the at least one nerve stimulation device is located on the cutting surface.

In some example embodiments, alone or in combination with any of the previous example embodiments, the device includes a third cutting link defining a third link distal end and a third link proximal end with the third link distal end being rotatably fixed to the second cutting apparatus link and the third link proximal end being rotatably fixed to the guide member.

In some example embodiments, alone or in combination with any of the previous example embodiments, the second cutting apparatus link is structured to be parallel with guide member in an instance the retractable cutting apparatus is in the deployed position.

In some example embodiments, alone or in combination with any of the previous example embodiments, the catheter includes a camera lumen. In some example embodiments, alone or in combination with any of the previous example embodiments, the device includes a camera structured to be positioned in the camera lumen.

In another example, a method of creating elongated incisions within a pericardium. The method includes placing a catheter within a human. The catheter includes at least one lumen, a longitudinal axis, a proximal end, and a distal end. The method also includes positioning an incision device within a pericardial cavity. The incision device is operably coupled to the distal end of the catheter. The incision device includes an incision channel defined along the incision device and the incision channel defines an open channel top and a channel bottom. The method further includes moving a retractable cutting apparatus disposed within the incision channel from a retracted position to a deployed position. In an instance in which the retractable cutting apparatus is in the retracted position, a first blade is disposed within the incision channel. In an instance in which the retractable cutting apparatus is in the deployed position, the first blade at least partially protrudes from the incision channel. The method also includes moving the retractable cutting apparatus along the pericardial cavity. An incision is made into a parietal layer of the pericardium upon movement of the retractable cutting apparatus along the pericardial cavity.

In some example embodiments, alone or in combination with any of the previous example embodiments, the retractable cutting apparatus includes a first cutting apparatus link defining a first link distal end and a first link proximal end with the first link distal end being rotatably fixed proximate to a distal end of the incision channel; a second cutting apparatus link defining a second link distal end and a second link proximal end with the second link distal end being attached to the first link proximal end of the first cutting apparatus link; and a guide member coupled to the second link proximal end, wherein the guide member is structured to move the first cutting apparatus link and the second cutting apparatus link and cause the retractable cutting apparatus to move between the retracted position and the deployed position.

In some example embodiments, alone or in combination with any of the previous example embodiments, the method includes moving the guide member in a direction of the second link with the retractable cutting apparatus being moved into the deployed position.

In some example embodiments, alone or in combination with any of the previous example embodiments, at least one of the first cutting apparatus link or the second cutting apparatus link is a blade. In some example embodiments, alone or in combination with any of the previous example embodiments, the first cutting apparatus link and the second cutting apparatus link are each a blade. In some example embodiments, alone or in combination with any of the previous example embodiments, the first cutting apparatus link includes a first blade. In some example embodiments, alone or in combination with any of the previous example embodiments, the second cutting apparatus link includes a secondary blade.

In some example embodiments, alone or in combination with any of the previous example embodiments, the first cutting apparatus link is a first blade including a cutting surface. In some example embodiments, alone or in combination with any of the previous example embodiments, the second cutting apparatus link is a secondary blade including a cutting surface.

In some example embodiments, alone or in combination with any of the previous example embodiments, in an instance in which the guide member is moved in a second direction opposite of the first cutting apparatus link and the second cutting apparatus link, the first link proximal end of the first cutting apparatus link and the second link distal end of the second cutting apparatus link are structured to move in a direction of the channel bottom to the retracted position.

In some example embodiments, alone or in combination with any of the previous example embodiments, the method includes moving the retractable cutting apparatus between the retracted position and the deployed position upon placement of the incision device within the pericardial cavity of the human body.

In some example embodiments, alone or in combination with any of the previous example embodiments, the creation of the at least one incision length through the pericardium causes a reduction in pressure exerted on a heart by the pericardium.

In some example embodiments, alone or in combination with any of the previous example embodiments, the incision length is created in at least one of a parietal layer or fibrous layer of the pericardium. In some example embodiments, alone or in combination with any of the previous example embodiments, the incision length is created in adipose tissue or fat deposits. In some example embodiments, alone or in combination with any of the previous example embodiments, the at least one incision length is along a length or circumference of only the parietal layer of the pericardium. In some example embodiments, alone or in combination with any of the previous example embodiments, the at least one incision length is made in the parietal layer from an anterior to a posterior of a heart. In some example embodiments, alone or in combination with any of the previous example embodiments, the at least one incision length is made in the parietal layer from a posterior base to an apex of a heart. In some example embodiments, alone or in combination with any of the previous example embodiments, the at least one incision length is made in the parietal layer from a posterior right atrium to an apex of a heart. In some example embodiments, alone or in combination with any of the previous example embodiments, the at least one incision length is made in the parietal layer from a left ascending aorta to an apex of a heart. In some example embodiments, alone or in combination with any of the previous example embodiments, the at least one incision length is made in the parietal layer from a right ascending aorta to an apex of a heart. In some example embodiments, alone or in combination with any of the previous example embodiments, the at least one incision length is made in the parietal layer transversely about a heart.

In some example embodiments, alone or in combination with any of the previous example embodiments, before creating the incision length, the method includes puncturing pericardial tissue and providing an access point into a pericardial space. In some example embodiments, alone or in combination with any of the previous example embodiments, before puncturing, the method includes providing subxiphoid access to the pericardium. In some example embodiments, alone or in combination with any of the previous example embodiments, before puncturing, the method includes providing transvascular access to the pericardium.

In some example embodiments, alone or in combination with any of the previous example embodiments, the method includes moving the retractable cutting apparatus from the deployed position to the retracted position before removing the incision device from the pericardium. In some example embodiments, alone or in combination with any of the previous example embodiments, the method includes puncturing into the pericardium via an introducer. In some example embodiments, alone or in combination with any of the previous example embodiments, after puncturing, the method includes inserting a guidewire into the pericardial cavity.

In some example embodiments, alone or in combination with any of the previous example embodiments, the method includes stabilizing the incision device within the pericardial cavity. In some example embodiments, alone or in combination with any of the previous example embodiments, the incision device is stabilized via one or more stabilization member. In some example embodiments, alone or in combination with any of the previous example embodiments, the one or more stabilizing members are structured to move between a stabilizing retracted position and a stabilizing deployed position.

In some example embodiments, alone or in combination with any of the previous example embodiments, the one or more stabilizing members are moved from the stabilizing retracted position to the stabilizing deployed position upon positioning of the catheter within the human body. In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures.

In some example embodiments, alone or in combination with any of the previous example embodiments, the method includes moving a sheath from a first position covering the incision channel to a second position that uncovers the incision channel. In some example embodiments, alone or in combination with any of the previous example embodiments, the sheath is structured to cover the incision channel in an instance in which the catheter is not positioned within human body. In some example embodiments, alone or in combination with any of the previous example embodiments, the one or more openings of the sheath are traversable along the catheter allowing an actuator to cause the one or more stabilizing members to laterally project through both the catheter and the sheath.

In some example embodiments, alone or in combination with any of the previous example embodiments, the method includes sterilizing the incision device. In some example embodiments, alone or in combination with any of the previous example embodiments, the incision device is sterilized via E-beam sterilization. In some example embodiments, alone or in combination with any of the previous example embodiments, the incision device is sterilized via gamma sterilization. In some example embodiments, alone or in combination with any of the previous example embodiments, the incision device is sterilized via ethylene oxide sterilization. In some example embodiments, alone or in combination with any of the previous example embodiments, the incision device is sterilized via autoclave sterilization.

In some example embodiments, alone or in combination with any of the previous example embodiments, the method includes determining a location of the incision device within the pericardial cavity via ultrasound. In some example embodiments, alone or in combination with any of the previous example embodiments, creating the at least one incision length is determined in response to a signal indicative of a reduction of restraint of the heart. In some example embodiments, alone or in combination with any of the previous example embodiments, creating the at least one incision length is determined in response to a signal indicative of a reduction of restraint of the heart; and repeating the creating of the at least one incision length.

In some example embodiments, alone or in combination with any of the previous example embodiments, after creating the at least one incision length, the method includes confirming a location of a distal end of the catheter device; and in response to a signal indicative of a reduction of restraint of the heart, repeating the steps of creating the at least one incision length, and confirming a location of the distal end.

In some example embodiments, alone or in combination with any of the previous example embodiments, the retractable cutting apparatus includes at least two blades. In some example embodiments, alone or in combination with any of the previous example embodiments, the retractable cutting apparatus includes an electrode. In some example embodiments, alone or in combination with any of the previous example embodiments, the retractable cutting apparatus includes at least one blade and at least one electrode. In some example embodiments, alone or in combination with any of the previous example embodiments, the electrode is structured to receive current and/or radio frequency energy to ablate, burn, vaporize, and/or separate tissue. In some example embodiments, alone or in combination with any of the previous example embodiments, the method includes providing an electrical current to the retractable cutting apparatus.

In some example embodiments, alone or in combination with any of the previous example embodiments, after an incision is formed within the pericardium, at least a portion of the retractable cutting apparatus extends through the incision. In some example embodiments, alone or in combination with any of the previous example embodiments, the retractable cutting apparatus is connected to an energy source connector that electrifies the cutting surface. In some example embodiments, alone or in combination with any of the previous example embodiments, the energy source connector is a wire connected to an energy source. In some example embodiments, alone or in combination with any of the previous example embodiments, the energy source is a battery or generator.

In some example embodiments, alone or in combination with any of the previous example embodiments, the method includes receiving a signal from at least one nerve detection device. In some example embodiments, alone or in combination with any of the previous example embodiments, the at least one nerve detection device is located on the catheter. In some example embodiments, alone or in combination with any of the previous example embodiments, the at least one nerve detection device is located adjacent the incision device. In some example embodiments, alone or in combination with any of the previous example embodiments, the at least one nerve detection device is located on the puncturing tip. In some example embodiments, alone or in combination with any of the previous example embodiments, the at least one nerve detection device is located on the cutting surface.

In some example embodiments, alone or in combination with any of the previous example embodiments, the method includes receiving a signal from at least one nerve stimulation device. In some example embodiments, alone or in combination with any of the previous example embodiments, the at least one nerve stimulation device is located on the catheter. In some example embodiments, alone or in combination with any of the previous example embodiments, the at least one nerve stimulation device is located adjacent the incision device. In some example embodiments, alone or in combination with any of the previous example embodiments, the at least one nerve stimulation device is located on the puncturing tip. In some example embodiments, alone or in combination with any of the previous example embodiments, the at least one nerve stimulation device is located on the cutting surface.

In another example embodiment, a medical device is provided. The medical device includes an incision device structured to be operably coupled to a distal end of a catheter. The incision device includes an incision channel defined along the incision device. The incision channel defines an open channel top and a channel bottom. The medical device also includes a retractable cutting apparatus disposed within the incision channel. The retractable cutting apparatus includes a first cutting apparatus link defining a first link distal end and a first link proximal end. The first link distal end is rotatably fixed proximate to a distal end of the incision channel. The retractable cutting apparatus also includes a second cutting apparatus link defining a second link distal end and a second link proximal end. The second link distal end is attached to the first link proximal end of the first cutting apparatus link. The retractable cutting apparatus further includes a guide member coupled to the second link proximal end. The guide member is structured to move the first cutting apparatus link and the second cutting apparatus link and cause the retractable cutting apparatus to move between a retracted position and a deployed position. The retractable cutting apparatus still further includes a first blade attached to the first link proximal end of the first cutting apparatus link. The first blade is structured to protrude from the incision channel in an instance in which the retractable cutting apparatus is in the deployed position. In an instance in which the guide member is moved in a first direction of the first cutting apparatus link and the second cutting apparatus link, the first link proximal end of the first cutting apparatus link and the second link distal end of the second cutting apparatus link are structured to move in a direction of the open channel top to the deployed position. In an instance in which the guide member is moved in a second direction opposite of the first cutting apparatus link and the second cutting apparatus link, the first link proximal end of the first cutting apparatus link and the second link distal end of the second cutting apparatus link are structured to move in a direction of the channel bottom to the retracted position.

In some example embodiments, alone or in combination with any of the previous example embodiments, the cutting apparatus is structured to be moveable between the retracted position and the deployed position upon placement of the incision device.

In some example embodiments, alone or in combination with any of the previous example embodiments, the second cutting apparatus link defines a first blade receiving aperture that is structured to receive the first blade in an instance in which the retractable cutting apparatus is in the retracted position.

In some example embodiments, alone or in combination with any of the previous example embodiments, upon movement to the deployed position, the first blade is structured to make an incision along a pericardium upon movement of the catheter.

In some example embodiments, alone or in combination with any of the previous example embodiments, at least one of the first cutting apparatus link and the second cutting apparatus link comprise at least one of a blade or an electrode.

In some example embodiments, alone or in combination with any of the previous example embodiments, the incision channel comprises a channel protrusion structured to prevent the first blade and the second blade from being parallel along the incision channel.

In some example embodiments, alone or in combination with any of the previous example embodiments, the channel protrusion causes an angle between the first blade and a secondary blade to be less than 180 degrees to assist the movement between the retracted position and the deployed position.

In some example embodiments, alone or in combination with any of the previous example embodiments, the first blade remains stationary along the incision channel in an instance in which the catheter is being moved.

In some example embodiments, alone or in combination with any of the previous example embodiments, the retractable cutting apparatus further includes a secondary blade structured to compliment the first blade during cutting.

In some example embodiments, alone or in combination with any of the previous example embodiments, the first blade defines a cutting surface and the secondary blade defines a secondary cutting surface and each of the cutting surface and the secondary cutting surface is structured to cut into the pericardium.

In some example embodiments, alone or in combination with any of the previous example embodiments, the cutting surface and the secondary cutting surface are structured to cut in opposite directions, such that a scissor effect or a glide effect occurs.

In some example embodiments, alone or in combination with any of the previous example embodiments, the first blade is rigidly attached to the first cutting apparatus link.

In some example embodiments, alone or in combination with any of the previous example embodiments, the secondary blade is rigidly attached to the secondary cutting apparatus link.

In some example embodiments, alone or in combination with any of the previous example embodiments, the first blade defines a blade height that is defined by the amount the first blade protrudes from the incision channel in the deployed position, wherein the blade height is adjustable.

In some example embodiments, alone or in combination with any of the previous example embodiments, the medical device also includes an introducer positioned near a distal end of the incision device.

In some example embodiments, alone or in combination with any of the previous example embodiments, the medical device also includes a guide member movement actuator attached to the guide member with the guide member movement actuator being manipulated to move the guide member.

In some example embodiments, alone or in combination with any of the previous example embodiments, the medical device is sterilized.

In some example embodiments, alone or in combination with any of the previous example embodiments, the medical device also includes a controller engaged to the incision device.

In some example embodiments, alone or in combination with any of the previous example embodiments, the controller is structured to provide at least one of movement of the blade between the retracted position and the deployed position, movement of the guide member, movement of the retractable cutting apparatus along the incision device, movement of the incision device, movement of the catheter, movement of the sheath between a covered position and an uncovered position, energy to the incision device, or movement of one or more stabilizing members.

In another example embodiment, a method of manipulating a medical device is provided. The method includes providing a medical device of any one of the previous claims engaged with a controller. The method also includes controlling at least one of movement of the blade between the retracted position and the deployed position, movement of the guide member, movement of the retractable cutting apparatus along the incision device, movement of the incision device, movement of the catheter, movement of the sheath between a covered position and an uncovered position, supply of energy to the incision device, or movement of one or more stabilizing members.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIGS. 1B and 1C are different enlarged cross-sectional views of the heart wall depicted in FIG. 1A illustrating layers of the heart wall and the pericardial cavity.

FIG. 2A is a medical device of an example embodiment with a sheath covering an incision channel in accordance with various embodiments.

FIG. 2B is a medical device of an example embodiment in the retracted position with the sheath uncovered in accordance with various embodiments.

FIG. 3A illustrates an example retractable cutting apparatus in the retracted position in accordance with various embodiments.

FIG. 3B illustrates the example retractable cutting apparatus moving between the retracted position and the deployed position in accordance with various embodiments.

FIG. 3C illustrates the retractable cutting apparatus in the deployed position in accordance with various embodiments.

FIGS. 4 and 5 illustrates an example medical device with different blade height in accordance with various embodiments.

FIG. 6 depicts an exemplary controller device for delivering the incision devices in accordance with various embodiments.

FIGS. 7A and 7B is an example guide member movement actuator used to manipulate the guide member in accordance with various embodiments.

FIG. 8 is another example embodiment of the retractable cutting apparatus in accordance with various embodiments.

FIGS. 9A-9C illustrate the medical device being deployed within the pericardium in accordance with various embodiments.

FIG. 10 illustrates another example retractable cutting apparatus in the deployed position in accordance with various embodiments.

FIG. 11 is a cross-section view of the example retractable cutting apparatus shown in FIG. 10 in accordance with various embodiments.

FIG. 12 is a simplified diagram of a multi-lumen catheter approach to the pericardial cavity in accordance with various embodiments.

FIG. 13 is a simplified diagram of an alternative multi-lumen catheter approach to the pericardial cavity in accordance with various embodiments.

FIG. 14 is a simplified diagram of a parietal layer incision length and cut path in accordance with various embodiments.

### DETAILED DESCRIPTION

The present disclosure provides for a catheter-based therapy referred to as transcatheter alleviation of pericardial restraint (TAPR) that can reduce pericardial restraint by incising or opening the pericardium with the intention of improving patient outcomes with heart dysfunction, for example, HFpEF or HFrEF and reducing HF readmissions related thereto. The present disclosure, in one example, provides a device with a concealed/medially-facing cutting surface for accessing and modifying a subject's pericardium for relieving pericardial restraint and/or resolving a heart dysfunction. The present disclosure further provides for methods of treating heart dysfunction using the presently disclosed device.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “pericardial restraint” includes any terms that signify impaired filling caused by excessive forces from the pericardium.

As used herein the phrase “cutting surface” is inclusive of one or more of an edge of a sharpened blade or the surface of an electrode structured to receive sufficient current or radio frequency energy (RF) to ablate, burn, vaporize, or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a catheter, and the application of a directional force sufficient to cut or separate the tissue, the force being substantially in a direction towards the proximal end of the catheter, for example, by pulling the catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an access point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “preserved ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “reduced ejection fraction” is inclusive of the ordinary and customary meaning to one of ordinary skill in medical and surgical arts, for example, impairment of ventricular filling or ejection of blood or both, with a clinical syndrome in which patients display left ventricular ejection fraction (LVEF) of 40% or less and are accompanied by progressive left ventricular dilatation and adverse cardiac remodeling and/or mitral valve dysfunction.

As used herein the phrase “heart dysfunction” is inclusive of the ordinary and customary meaning to one of ordinary skill in medical and surgical arts, for example, heart failure or congestive heart failure.

As used herein the phrase “incision device” is inclusive of a device with a cutting surface, for example an edge of a blade or a surface of an electrode.

As used herein the phrase “pericardial incision assembly” and “incision assembly” are used interchangeable and refer to an assemblage that includes an incision device.

As used herein the phrase “catheter” or “catheter device” is inclusive of a catheter structured with at least one lumen comprising a medical instrument, device, or component thereof, for example, an incision device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein the phrase “puncturing tip” is inclusive of an atraumatic object suitable for puncturing or penetrating tissue without substantial trauma to or bleeding from the vicinity of the picture or penetration.

Several exemplary transection devices for making incisions through the pericardial membrane or parietal layer of the pericardium are described herein. These examples share the characteristic that they are deployed intravascularly through the right atrial appendage (RAA), Inferior Vena Cava (IVC), Superior Vena Cava (SVC), or via a subxiphoid approach.

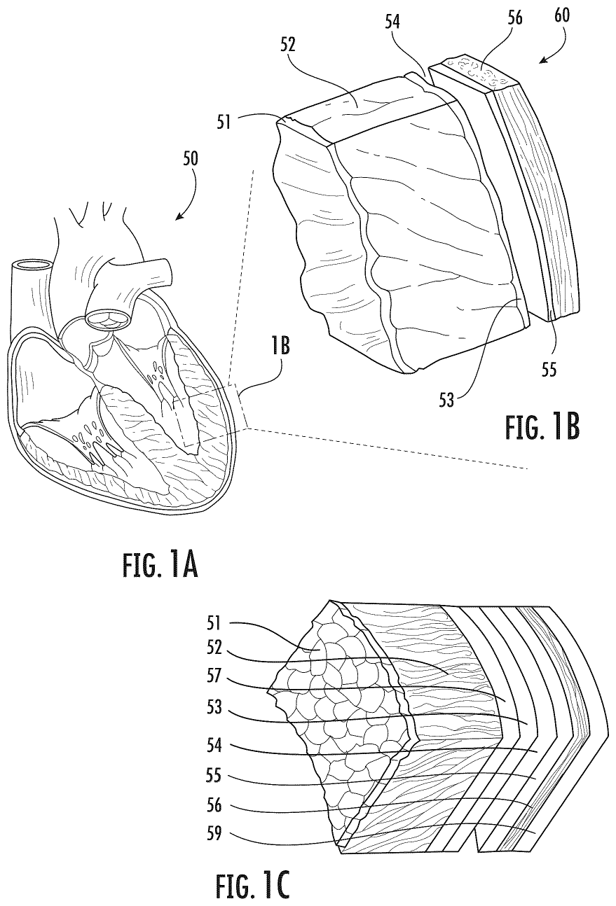

With reference to FIGS. 1A, 1B, 1C, and sections 1B, 1C, layers of a heart wall of a heart 50, with pericardium 60, from inside-out, being the endocardium 51, the myocardium 52, epicardial adipose tissue 57, the visceral layer 53, the pericardium cavity 54, the parietal layer 55, the fibrous pericardium 56, and the pericardial adipose tissue 59 are depicted. In one example, the presently disclosed devices are structured for introduction to the pericardium cavity 54 and for cutting tissue layers generally disposed adjacent to and including adipose tissue within and outside the pericardial cavity and superficial to the visceral layer 53 of the pericardium 60.

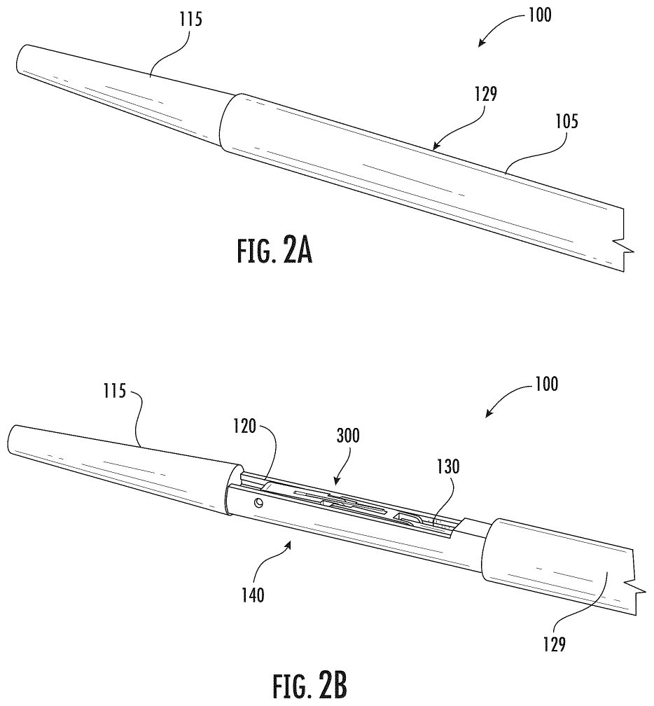

Referring now to FIGS. 2A and 2B, in one example the presently disclosed medical device 100 comprises a flexible catheter 129 comprising a distal end, at least one lumen, and a longitudinal axis; an incision device 140 coupled to the distal end of the catheter; and an introducer 115 coupled to and projecting from the incision device 140. The catheter may be steerable. In one example, the introducer 115 comprises a puncturing tip. The puncturing tip of the introducer 115 is structured to provide enough force to the introducer to puncture human tissue to allow access to the pericardium.

In one example, at least a portion of the flexible catheter 129 tip is radiopaque. In one example, at least a portion of the incision device 140 is radiopaque. In one example, at least a portion of the introducer 115 tip is radiopaque.

As shown in FIG. 2A, the medical device 100 may also include a sheath 105 structured to moveably cover the incision channel 120. The sheath 105 is structured to cover the incision channel 120 in an instance in which the catheter 129 is not positioned within human body. Once the catheter has been positioned (e.g., the incision device is positioned where the cut in the parietal layer 55 of the pericardium 60 is to begin), the sheath 105 may be actuated to uncover the incision channel 120. In some embodiments, the sheath 105 may not be moveable and merely has a slit to allow the knife to protrude. The sheath 105 can also hold the one or more stabilizing members in the stabilizing retracted position during movement (e.g., the sheath moves to uncover the stabilizing member(s) upon placement in the pericardium cavity 54).

The incision device 140 defines an incision channel 120 that is structured to receive a retractable cutting apparatus 300. The retractable cutting apparatus 300 can be moved along the incision channel 120 via an internal actuator (e.g., connected to the guide member 130) to move the retractable cutting apparatus 300 between a deployed position and a retracted position.

FIG. 2B illustrates the blade of the retractable cutting apparatus 300 in the retracted position. As discussed herein, the blade is structured to move between a retracted position and a deployed position. In the retracted position, the blade is disposed within the incision channel (e.g., not protruding from the incision channel). In the deployed position, the blade at least partially protrudes from the incision channel.

Part or all of the medical device 100 may be sterilized for use. The medical device may be sterilized using various sterilizing techniques, such as E-Beam sterilization, gamma sterilization, ethylene oxide sterilization, autoclave sterilization, including aseptic sterilization and aseptic manufacturing/packaging, and/or the like. Additionally, one or more materials used in the medical device may have anti-bacterial characteristics.

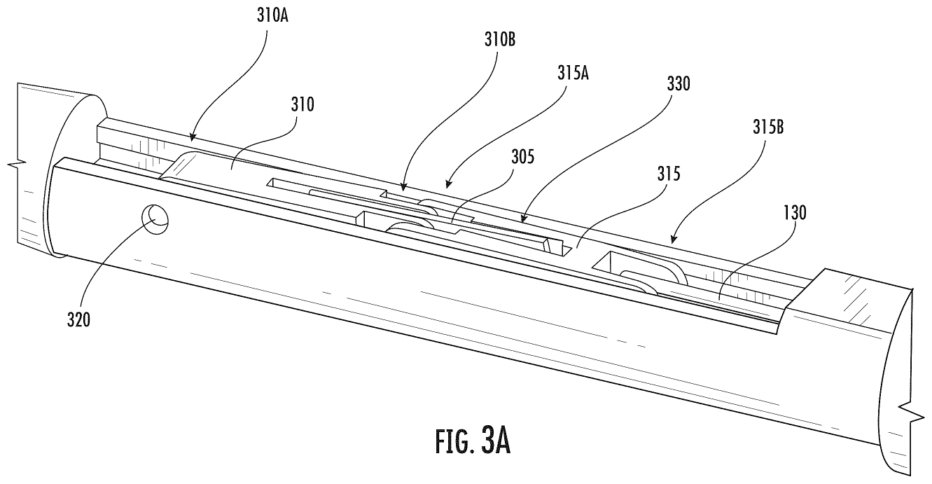

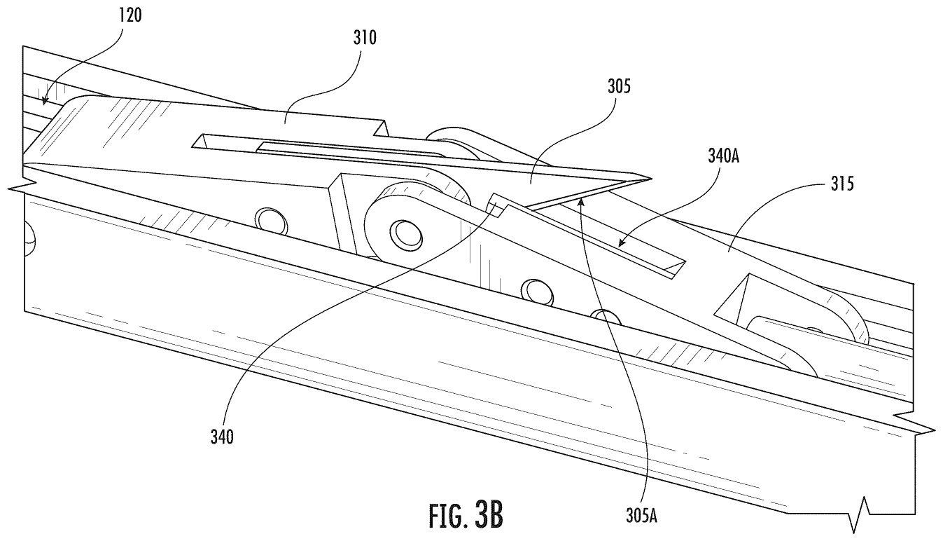

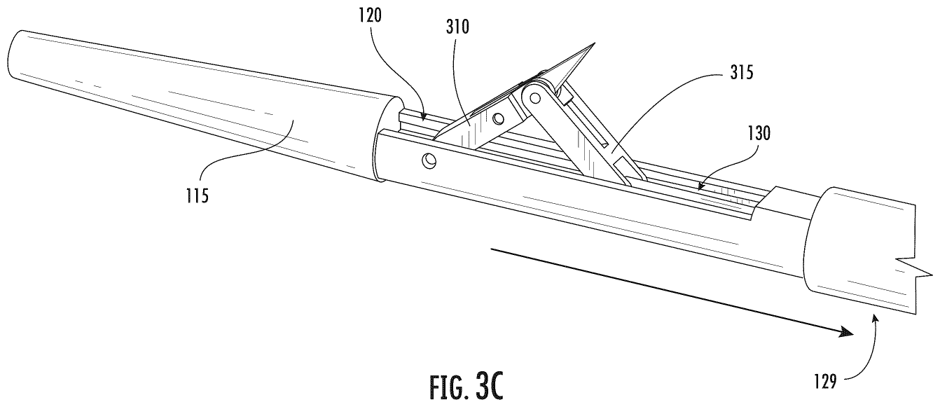

Referring now to FIGS. 3A-3C, an example retractable cutting apparatus 300 is shown. As discussed above in reference to FIG. 2, the retractable cutting apparatus 300 is movably positioned within the incision channel 120 of the incision device between a retracted position (FIG. 3A) and a deployed position (FIG. 3C). FIG. 3A illustrates an example retractable cutting apparatus 300 in the retracted position. FIG. 3B illustrates the example retractable cutting apparatus 300 moving between the retracted position and the deployed position. FIG. 3C illustrates the retractable cutting apparatus 300 in the deployed position.

The retractable cutting apparatus 300 includes a first cutting apparatus link 310, a second cutting apparatus link 315, a first blade 305, and a guide member 130. The first cutting apparatus link 310 defines a first link distal end 310A and a first link proximal end 310B. The first cutting apparatus link 310 is rotatably attached to the incision channel 120 proximate to the first link distal end 310A (e.g., at point 320). The first link distal end 310A of the first cutting apparatus link 310 is fixed in the direction along the incision device (e.g., from movement along the incision channel 120). The first cutting apparatus link 310 can rotatably move in the direction generally perpendicular to the fixed direction along the incision device.

The second cutting apparatus link 315 defines a second link distal end 315A and a second link proximal end 315B. The second cutting apparatus link 315 is rotatably attached to the first cutting apparatus link 310 proximate to the second link distal end 315A of the second cutting apparatus link 315 and the first link proximal end 310B of the first cutting apparatus link 310.

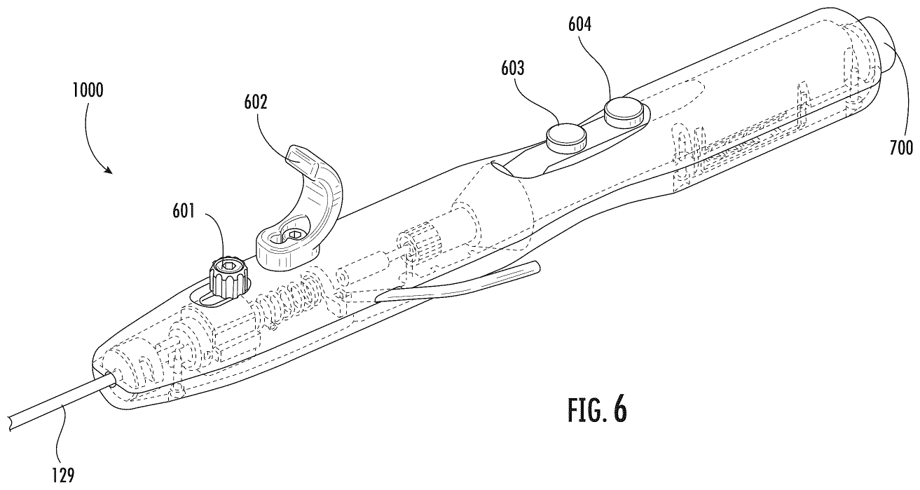

The second cutting apparatus link 315 may be operably coupled to the guide member 130 proximate to the second link proximal end 315B. As such, the guide member 130 is received by a slot within the second cutting apparatus link 315 and rotatably attached via a pin or the like. Various attachments methods may be used to allow for the guide member to be rotatably attached to the second cutting apparatus link 315. In some embodiments, as discussed below in reference to FIG. 6, the retractable cutting apparatus 300 may have one or more intermediate links between the second cutting apparatus link 315 and the guide member, however, in such an instance the guide member 130 is still operably coupled to the second cutting apparatus link 315 (e.g., the movement of the guide member 130 causes movement of the second cutting apparatus link 315).

The first blade 305 is attached to the first cutting apparatus link 310. The first blade 305 is rigidly attached to the first cutting apparatus link 310. For example, as shown in FIG. 3B, the first cutting apparatus link 310 defines a slot that receives the first blade 305 and the first blade is attached to the first cutting apparatus link 310. While a pin is shown as the attachment method of the first blade, various other attachment methods may be used. The first blade 305 protrudes from the first link proximal end 310B of the first cutting apparatus link 310.

The first blade 305 may have a first blade cutting surface 305A that is positioned to cut tissue upon being moved within the human body in an instance in which the first blade 305 is in the deployed position. The first blade cutting surface 305A of the first blade 305 when laterally projected (e.g., in the deployed position), faces away from the distal end and towards the proximal end of the catheter. The first blade 305 may define a puncture tip that is a sharp edge structured to puncture pericardium tissue when being rotated into the deployed position. The first blade 305 is structured to have a sufficient force to puncture pericardial tissue in an instance in which the retractable cutting apparatus 300 is moved from the retracted position to the deployed position.

The second cutting apparatus link 315 may define a blade receiving channel 330 that is structured to receive the first blade 305 in an instance in which the retractable cutting apparatus 300 is in the retracted position. The blade receiving channel 330 is an aperture that extends from the second link distal end 315A in the direction of the second link proximal end 315B. The length of the blade receiving channel 330 may be based on the length of the first blade 305 (e.g., the blade receiving channel 330 may be slightly longer than the first blade to allow for the entire first blade 305 to be disposed within the blade receiving channel 330).

A second blade 340 may be rigidly attached to the second cutting apparatus link 315. The second blade 340 may be disposed within the blade receiving channel 330 of the second cutting apparatus link 315. As shown in FIG. 3B, the second blade 340 is positioned along the blade receiving channel 330. The second blade 340 may have a second blade cutting surface 340A that is positioned to cut tissue upon being moved within the human body in an instance in which the retractable cutting apparatus 300 is in the deployed position. The second blade cutting surface 340A of the second blade 340 vertically protrudes from the second cutting apparatus link 315 (e.g., vertically protrudes from the blade receiving channel 330). The first blade cutting surface 305A and the second blade cutting surface 340A are positioned to face in opposing directions in the deployed position, such that the first cutting surface is generally facing downward (e.g., in the direction of the incision channel) and the second blade cutting surface 340A is generally facing upward (e.g., in the direction opposite of the incision channel). As such, the first blade cutting surface 305A and the second blade cutting surface 340A are structured to provide a scissor effect or glide effect on the tissue of the parietal layer 55 of the pericardium 60. In various embodiments, the scissor effect may include the first blade cutting surface 305A and the second blade cutting surface 340A moving relative to one another during the cutting process. In various embodiments, the glide effect includes the first blade cutting surface 305A and the second blade cutting surface 340A being fixed relative to one another during the cutting process. For example, the first blade cutting surface 305A and the second blade cutting surface 340A glide through the tissue substantially without movement of either the first blade cutting surface 305A or the second blade cutting surface 340A.

As shown in FIG. 3A, in the retracted position, each of the first cutting apparatus link 310, the second cutting apparatus link 315, and the guide member 130 are positioned along a first plane (e.g., within the incision channel 120). The first blade 305 of the first cutting apparatus link 310 is received by the second cutting apparatus link 315 as discussed above. In the retracted position, the sheath 105 shown in FIG. 2 may be moved over the incision channel 120 covering the retractable cutting apparatus 300 (e.g., for transport and positioning within the pericardium cavity 54 to avoid inadvertent cuts).

As the guide member 130 moves in the direction of the second cutting apparatus link 315, the retractable cutting apparatus 300 begins to move from the retracted position to the deployed position. As the guide member 130 moves in the direction of the second cutting apparatus link 315, the connection between the first cutting apparatus link 310 and the second cutting apparatus link 315 moves upward from the incision channel (e.g., in the direction of an open channel top of the incision channel 120). A v-shape begins to form between the first cutting apparatus link 310 and the second cutting apparatus link 315 (as shown in FIG. 3B). The first blade 305 begins to extend from the blade receiving channel 330 to expose the first blade cutting surface 305A. The second blade 340 is also exposed.

A linkage distance is defined between the first link distal end 310A of the first cutting apparatus link 310 and the second link proximal end 315B of the second cutting apparatus link 315. As such, the linkage distance is greater in an instance in which the retractable cutting apparatus 300 is in the retracted position than an instance in which the retractable cutting apparatus 300 is in the deployed position.

The guide member 130 is structured to move a specific distance along the incision channel based on the desired blade height. The blade height is defined by the height of the tip of the first blade 305 from the open channel top of the incision channel 120 (e.g., how much the first blade protrudes from the incision channel 120). The blade height is adjustable. As discussed herein, the guide member may be connected to a lead screw that is structured to change the distance in which the guide member can move in the direction of the second cutting apparatus link 315, such that the blade height is changed (e.g., the farther the guide member can move in the direction of the second cutting apparatus link, the taller the blade height).

The guide member 130 may be positioned within a lumen of the catheter (e.g., a guidewire lumen). The guide member 130 may extend through the given lumen of the catheter and extend into the controller of the catheter as discussed herein. The controller may include a guide member movement actuator that is attached to the guide member. The guide member movement actuator is manipulated to move the guide member.

FIG. 3C illustrates an example retractable cutting apparatus 300 in a deployed position. Upon moving the retractable cutting apparatus 300 into the deployed position, the catheter is moveable along the pericardium cavity 54 to allow an incision to be made. The incision may define an incision length that is defined as the length in which the catheter is moved in an instance in which the retractable cutting apparatus 300 is in the deployed position. The incision may be linear due to the movement of the catheter. The retractable cutting apparatus 300 remains generally stationary relative to the catheter while the catheter is moving.

As the guide member 130 moves in the direction opposite of the second cutting apparatus link 315, the retractable cutting apparatus 300 begins to move to the retracted position. As the guide member 130 moves in the direction opposite of the second cutting apparatus link 315, the connection between the first cutting apparatus link 310 and the second cutting apparatus link 315 moves downward from the incision channel (e.g., in the direction of a bottom of the incision channel 120). The first blade 305 is received by the blade receiving channel 330. Once back to the retracted position, each of the first cutting apparatus link 310, the second cutting apparatus link 315, and the guide member 130 are positioned along a first plane (e.g., within the incision channel 120). Upon retraction, the sheath 105 shown in FIG. 2A may be moved to cover the incision channel for removal from the human body. One or more components of the medical device may be radiopaque to allow the medical device to be seen within the human body using ultrasound. In various embodiments, portions of the retractable cutting apparatus, catheter, and/or introducer may be radiopaque.

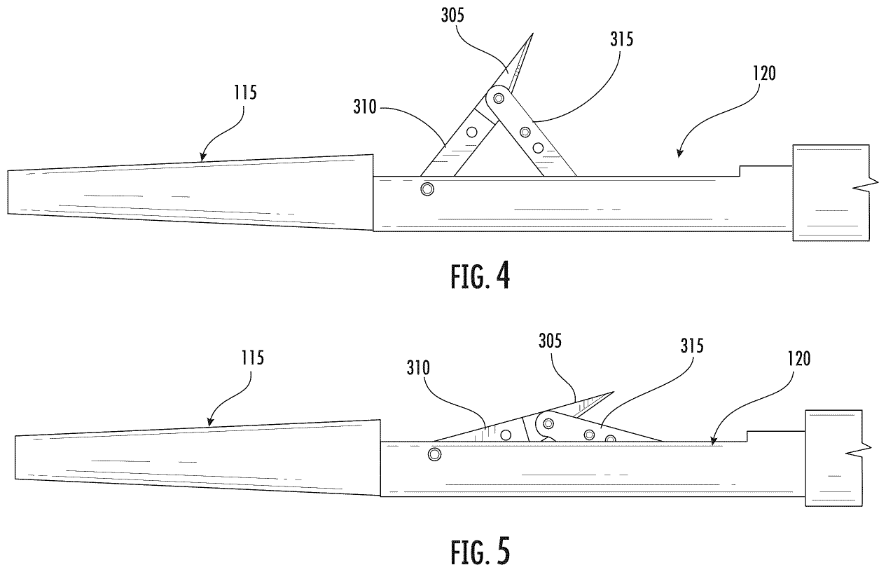

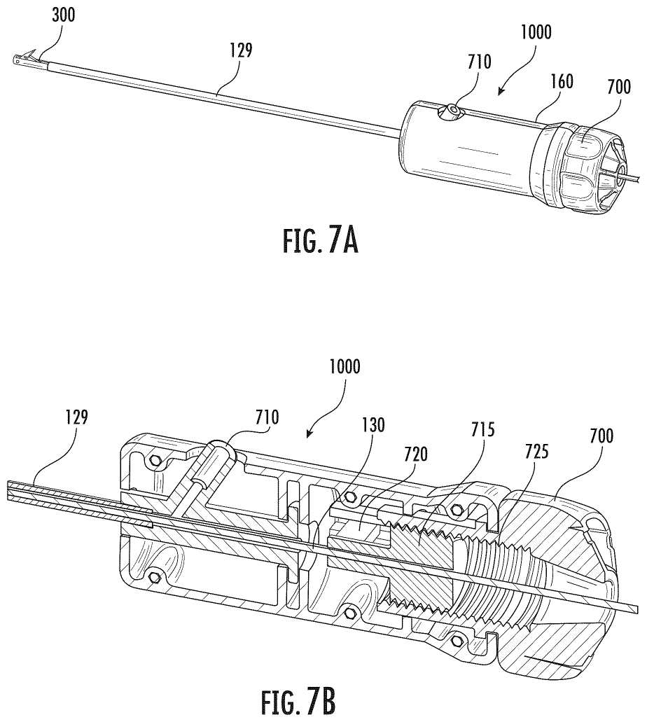

FIGS. 4 and 5 illustrate different blade projection heights and angles from the outer surface of the flexible catheter 129 (or the amount of protrusion of the first blade 305 from the incision channel 120) that are configurable for the retractable cutting apparatus 300. The blade height may be determined by the movement range of the guide member 130. The blade height may be adjusted based on the thickness of the tissue to be cut. As shown, the angle of the blade may also be adjusted, as the blade height of FIG. 4 results in the blade pointing more laterally than the blade height of FIG. 5. The blade height can be adjusted via the actuating knob 700 discussed below in reference to FIGS. 7A and 7B. The range of motion of the threaded screw 715 (e.g., the range of motion is based on the position of the screw stopper 720) can cause the blade height to be adjusted.

With reference to FIG. 6, the controller 1000 is shown as a handle 160. The controller may have multiple actuating knobs 601, 602, 700 and actuating buttons 603, 604 for controlling the catheter 129 and the various components of the medical device 100 (e.g., actuating the sheath 105, actuating stabilizing members 910). The actuating knob 700 may correspond to the actuating knob 700 discussed below in reference to FIGS. 7A and 7B. Knob 601 may be structured to rotate to extend or retract the flexible catheter 129. Knob 602 may be engaged to activate one or more components on the medical device (e.g., actuating the stabilizing member(s)). Similarly, the actuating buttons 603, 604 may be engaged to activate various components of the catheter (e.g., the device may use RF electrode cutting as well and the actuating buttons 603, 604 may be used). Various other controllers may be contemplated that allow for the retractable cutting apparatus to be deployed and retracted.

In one example, the controller 1000 allows the operation of various potential operations of the incision device 140, including biasing/clamping the distal blade open and closed, which can be achieved by an appropriate mechanism structured to pull/push a rod. In one example, there is a mechanism used to release/retrieve balloons/nitinol components that function to stabilize and apply counterpressure for the incision device 140 and its components, including the cutting surface. Controller 1000 may comprise one or buttons used to operate and control the electrosurgical features of the device.

As shown in FIGS. 7A and 7B, the controller 1000 may include a handle 160 with an actuating knob 700. The controller 1000 may be integrated with the example embodiment discussed above in reference to FIG. 6, such that the handle 160 includes one or more buttons or controls discussed above. The controller 1000 shown in FIG. 7A may have a handle 160 with an actuating knob 700. The controller 1000 also includes a liquid inlet 710 that is structured to allow a liquid, such as a saline flush and/or contrast substance, to be pass through the catheter 129 and into the human body. The controller 1000 is connected to the catheter 129 to operate the catheter 129 and/or the incision device 140.

A method of manipulating medical device 100 would include providing medical device 100 engaged with a controller (e.g., controller 1000 of FIG. 6 or FIG. 7A) and controlling, for example, manipulating any one or combination of one or more actuating knobs (e.g., actuating knobs 601, 602 of FIG. 6 or actuating knob 700 of FIG. 7A) and one or more actuating buttons (actuating buttons 603, 604 of FIG. 6) to provide at least one of movement of the blade between the retracted position and the deployed position, movement of the guide member, movement of the retractable cutting apparatus along the incision device, movement of the incision device, movement of the catheter, movement of the sheath between a covered position and an uncovered position, supply of energy to the incision device, movement of one or more stabilizing members and/or the like with controller 1000. The aforementioned method is applicable to any previously disclosed medical devices.

As shown in FIG. 7B, the actuating knob 700 is rotatably attached to the handle 160, such that the actuating knob 700 is rotated relative to the handle 160. The actuating knob 700 includes an internal threading 725 that is structured to receive an internal screw 715 with the internal screw moving based on the rotation of the actuating knob 700. The internal screw 715 is attached to the guide member 130, such that the movement of the internal screw 715 causes the guide member 130 to also move, which subsequently moves the retractable cutting apparatus 300 between the retracted position and the deployed position.

As the actuating knob 700 is turned, the threaded screw 715 moves along the longitudinal axis (e.g., along the internal threading 725). In an instance in which the actuating knob 700 is moved in the clockwise direction, the threaded screw 715 moves in the direction opposite of the catheter 129, causing the guide member 130 to also move in the direction of the threaded screw 715 (e.g., in the direction opposite of the distal end of the catheter 129). The movement of the guide member 130 in the direction opposite the catheter 129 causes the retractable cutting apparatus 300 to be moved from the deployed position to the retracted position.

In an instance in which the actuating knob 700 is rotated in the counterclockwise direction, the threaded screw 715 moves in the direction of the catheter 129 (e.g., in the direction of the distal end of the catheter 129) causing the guide member 130 to move in the direction of the distal end of the catheter 129 (e.g., in the direction of the retractable cutting apparatus 300). The movement of the guide member 130 in the direction opposite the catheter causes the retractable cutting apparatus 300 to be moved from the retracted position to the deployed position.

The threaded screw 715 has a screw stopper 720 that stops the movement of the threaded screw 715 in the direction opposite the catheter 129. As such, the screw stopper 720 is positioned to hit an internal protrusion of the handle 160 once the retractable cutting apparatus is moved into the deployed position. In various embodiments, the range of movement of the threaded screw 715 is determined by the screw stopper 720, such that the threaded screw 715 does not travel along the entire internal threading 725. In various embodiments, the screw stopper 720 may be adjustable to change the angle or position of the blade upon deployment, as shown in FIGS. 4 and 5 discussed above.

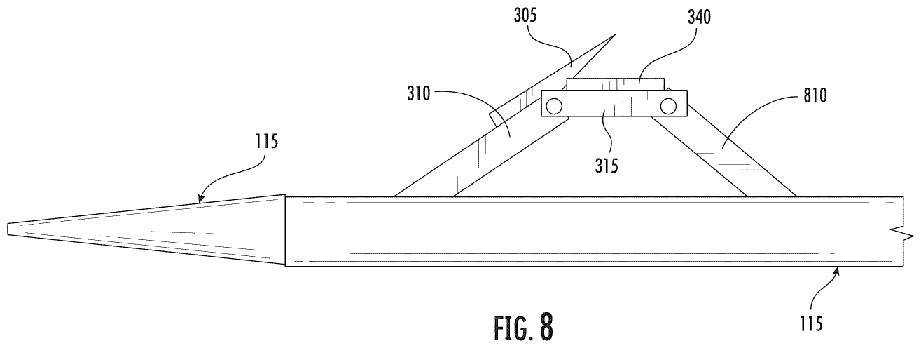

FIG. 8 is another example embodiment of retractable cutting apparatus 300. As shown, the retractable cutting apparatus 300 may include a third cutting apparatus link 810 that is positioned between the second cutting apparatus link 315 and the guide member 130. The guide member 130 is still structured to move along longitudinal axis (e.g., along the incision channel 120) as discussed above in reference to a two cutting apparatus link apparatus.

In an instance in which the guide member 130 is moved in the direction of the third cutting apparatus link 810 (and along the direction of the second cutting apparatus link 315 as above), the retractable cutting apparatus is moved to the deployed position. As the guide member 130 moves in the direction of the third cutting apparatus link 810, the connection between the first cutting apparatus link 310 and the second cutting apparatus link 315 and the connection between the second cutting apparatus link 315 and the third cutting apparatus link 810 each move laterally from the incision channel (e.g., in the direction of an open channel top of the incision channel 120).

In an instance, as shown above, in which the first cutting apparatus link 310 and the third cutting apparatus link 810 are equal length, the second cutting apparatus link 315 may remain approximately parallel to the incision channel. In some instances, one of the first cutting apparatus link 310 and the third cutting apparatus link 810 may be different lengths and the second cutting apparatus link 315 may be slanted (e.g., the second cutting apparatus link 315 may not be parallel to the incision channel). For example, the third cutting apparatus link 810 may be slightly shorter than the first cutting apparatus link 310 resulting in the second blade 340 be slanted to promote cutting by the retractable cutting apparatus 300.

Just as discussed above, the first blade 305 is disposed within the blade receiving channel of the second cutting apparatus link 315 in the retracted position and as the first cutting apparatus link moves in the lateral direction, the first blade 305 is exposed.

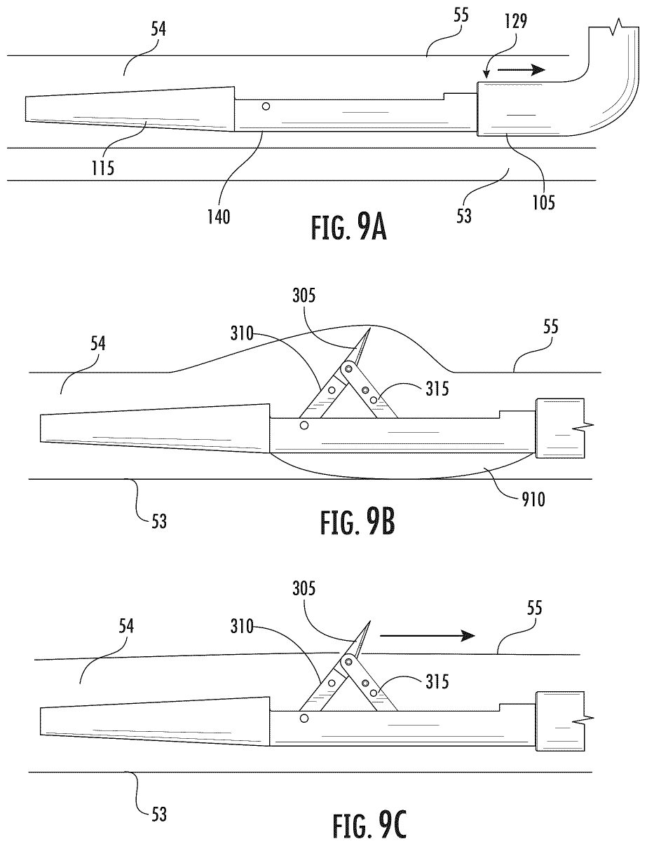

FIGS. 9A-9C illustrate medical device 100 being deployed. In one example, device 100 is deployed subxiphoidally, e.g., through percutaneous access achieved by directing a needle into the patient under the xiphoid process towards the pericardial cavity. In another example, device 100 is deployed intravascularly thru the right ventricle. The medical device 100 can be advanced through a subject's pericardium cavity 54 in undeployed configuration with the first blade 305 in the retracted position within the incision channel. The initial access to the pericardium cavity 54 may be achieved via the introducer 115. As the medical device is positioned within the pericardium cavity 54, the sheath 105 may be covering the incision channel. As the catheter is positioned within the pericardium cavity 54 at the location of the incision, the sheath 105 may be removed, as shown in FIG. 9A. The sheath may uncover the incision device 140 that is still in the retracted position.

As shown in FIG. 9B, the medical device 100 may have one or more stabilizing members 910. The stabilizing member 910 may be independently user controlled by advancing an actuating wire distally toward introducer 115 tip that laterally extends stabilizing member(s) 910 a distance from medical device 100. In one example, two or more stabilizing members 910 are positioned radially about the assembly. In one example, two or more stabilizing members 910 are positioned radially about the assembly about 120 degrees apart. In one example, the two or more stabilizing members 910 are offset longitudinally from the cutting surface to minimize or eliminate pushing the device through the newly cut slit in the pericardium just as it is formed. Stabilizing members 910 can be flexible rods or strip, or inflatable structures, such as balloons that can be inflated with air or liquid (saline).

The one or more stabilizing members 910 are structured to move between a stabilizing retracted position and a stabilizing deployed position upon positioning of the catheter within the human body.

The one or more stabilizing members 910 may be moved to the stabilizing retracted position after the blade has punctured the parietal layer 55 of the pericardium 60. For example, the stabilizing member(s) 910 may be structured to deploy to stabilize the catheter while the blade is making the cut into the tissue. After the initial cut into the parietal layer 55 of the pericardium 60 has been made, the stabilizing member(s) 910 may be moved to the stabilizing retracted position to allow the catheter to be moved along the pericardium cavity.

The retractable cutting apparatus 300 may be moved from the retracted position to the deployed position as discussed herein. The retractable cutting apparatus 300 uses the puncture tip of the first blade 305 to puncture the pericardial tissue to be cut. After the first blade 305 punctures the pericardial tissue, the incision can be made along the pericardium tissue of the parietal layer 55. In an instance in which the medical device 100 has stabilizing member(s) 910, the stabilizing member(s) 910 may be moved to the stabilizing retracted position to allow for the catheter to be moved. The tip of first blade 305 can serve as a radiopaque marker indicating ‘bunching up’ of tissue, for example, due to excessive pull before a through-cut is achieved. In one example, the back silhouette of the blade may be selectively insulated (as it may be activated to achieve the exit puncture) to avoid ablation damage to neighboring anatomy.

Upon puncture of the pericardium tissue (e.g., into the parietal layer 55) by the first blade, the catheter is moved along the pericardium cavity 54 to cause the incision for the incision length intended. The first blade cutting surface 305A of the first blade 305 and the second blade cutting surface 340A of the second blade 340 perform the cutting using the scissor effect or glide effect discussed above. The incision is a linear incision along the parietal layer 55. Upon moving the catheter the length of the intended incision, the retractable cutting apparatus is moved back to the retracted position and the sheath 105 can be moved to cover the incision channel. The catheter can then be removed from the human body without having any blades exposed.

In one example, at least one incision length is made in the pericardium of a heart (e.g., the incision is made in the parietal layer 55). The at least one incision lengths, in a heart with a dysfunction treatable with the present method, may cause the pericardium to separate radially about the cut line, without the need for removal of pericardial tissue. Other incision lengths and paths may be employed. Combinations of incision lengths and paths, and combination of incision lengths and paths with one or more of partial removal of pericardium, drainage, and other pericardial treatments can be employed.

In one example, creating at least one incision length is determined in response to a signal indicative of a reduction of restraint of the heart. In one example, creating at least one incision length is determined in response to a signal indicative of a reduction of restraint of the heart; and repeating the creating of at least one incision length. In one example, the presently disclosed method further comprises, after creating the at least one incision length, confirming a location of a distal end of the catheter device; and in response to a signal indicative of a reduction of restraint of the heart, repeating the steps of creating the at least one incision length, and confirming a location of the distal end.

In one example, a puncture to deliver a guidewire into the pericardial space is performed through heart tissue. When a transvascular approach through the RAA, IVC, or SVC is employed, a closure device may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

The following exemplary occlusion descriptions relate to a transvascular approach through the RAA, IVC, or SVC using one of the aforementioned transection devices. In one example, a puncturing tip 115 delivers a wire into the pericardial space through heart tissue. A closure or occlusion device is introduced for hemostasis during the procedure. The closure or occlusion device in one example includes outward or radially directed splines deployed in an expanded configuration. When the guide catheter is removed, the splines or radial members of the closure device contract inwardly towards the unstressed state of the transection device in order to close and seal the opening. The closure device is designed such that a pericardial cutting device can pass through and into the pericardial space.

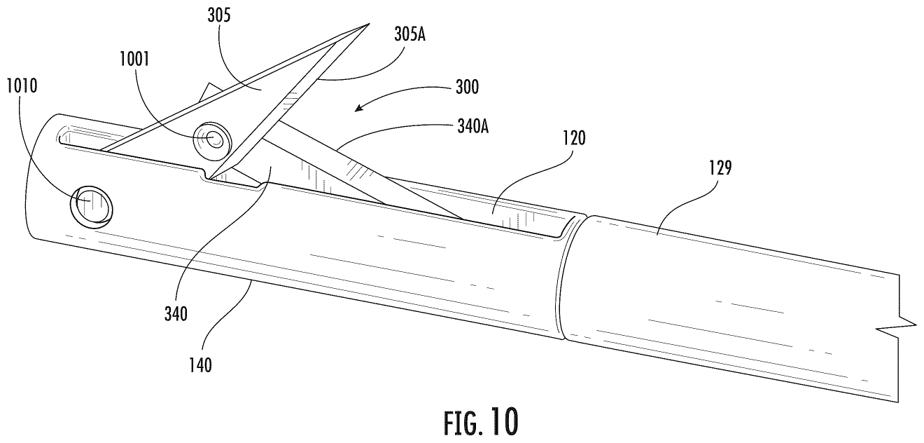

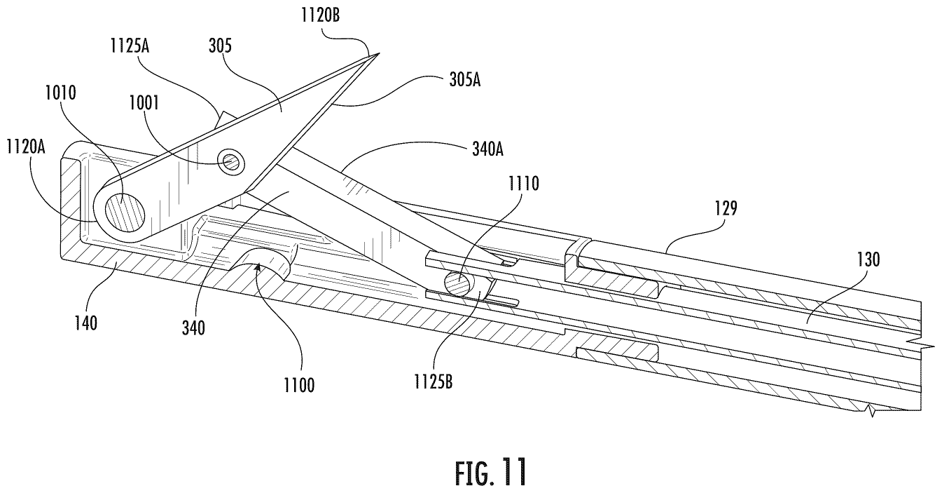

FIG. 10 illustrates another example retractable cutting apparatus in the deployed position in accordance with various embodiments. As shown, the first cutting apparatus link may be the first blade 305 and the second cutting apparatus link may be the second blade 340. As such, the first blade 305 and the second blade 340 are rotatably attached at point 1001 (e.g., the first blade 305 and the second blade 340 may be attached by a hypotube to create a rivet join). As with other embodiments, the retractable cutting apparatus 300 is movably positioned within the incision channel 120 of the incision device between a retracted position and a deployed position. FIGS. 10 and 11 each illustrate the retractable cutting apparatus 300 in the deployed position.

In various embodiments, the retractable cutting apparatus 300 includes a first blade 305, a second blade 340, and a guide member 130. The first blade 305 defines a first blade distal end 1120A and a first blade proximal end 1120B. The first blade 305 is rotatably attached to the incision channel 120 proximate to the first blade distal end 1120A (e.g., at point 1010). The first blade distal end 1120A of the first blade 305 is fixed in the axis along the incision device (e.g., from movement along the incision channel 120). The first blade 305 can rotatably move in the direction generally perpendicular to the fixed direction along the incision device.

The second blade 340 defines a second blade distal end 1125A and a second blade proximal end 1125B. The second blade 340 is rotatably attached to the first blade 305 proximate to the second blade distal end 1125A of the second blade 340 (e.g., attached at point 1001).

The second blade 340 may be operably coupled to the guide member 130 proximate to the second blade proximal end 1125B (e.g., attached at point 1110). As such, the guide member 130 is attached to the second blade 340 via a pin or the like. Various attachments methods may be used to allow for the guide member to be rotatably attached to the second blade 340.

The first blade 305 may have a first blade cutting surface 305A that is positioned to cut tissue upon being moved within the human body in an instance in which the first blade 305 is in the deployed position. The first blade cutting surface 305A of the first blade 305 when laterally projected (e.g., in the deployed position), faces away from the distal end and towards the proximal end of the catheter. The first blade 305 may define a puncture tip that is a sharp edge structured to puncture pericardium tissue when being rotated into the deployed position. The first blade 305 is structured to have a sufficient force to puncture pericardial tissue in an instance in which the retractable cutting apparatus 300 is moved from the retracted position to the deployed position.

A second blade 340 may have a second blade cutting surface 340A that is positioned to also cut tissue upon being moved within the human body in an instance in which the retractable cutting apparatus is in the deployed position. The first blade cutting surface 305A and the second blade cutting surface 340A are positioned to face in opposing directions in the deployed position, such that the first cutting surface is generally facing downward (e.g., in the direction of the incision channel) and the second blade cutting surface 340A is generally facing upward (e.g., in the direction opposite of the incision channel). As such, the first blade cutting surface 305A and the second blade cutting surface 340A are structured to provide a scissor cutting effect on the tissue of the pericardium. In various embodiments, the first blade cutting surface 305A and the second blade cutting surface 340A may be substantially locked into place in the deployed position, such that the first blade cutting surface 305A and the second blade cutting surface 340A are structured to provide a “glide” effect along the tissue.

In the retracted position, each of the first blade 305, the second blade 340, and the guide member 130 are positioned generally along a first plane (e.g., within the incision channel 120). The incision channel 120 may include a channel protrusion 1100 structured to prevent the first blade 305 and the second blade 340 from being parallel along the incision channel 120. The channel protrusion 1100 causes the angle between the first blade 305 and the second blade 340 to be less than 180 degrees to assist the movement between the retracted position and the deployed position.

As the guide member 130 moves in the direction of the second blade 340, the retractable cutting apparatus 300 begins to move from the retracted position to the deployed position. As the guide member 130 moves in the direction of the second blade 340, the connection (e.g., at point 1001) between the first blade 305 and the second blade 340 is caused to move upward from the incision channel (e.g., in the direction of an open channel top of the incision channel 120). A v-shape begins to form between the first blade 305 and the second blade 340 (as shown in FIGS. 10 and 11). The first blade cutting surface 305A of the first blade 305 begins to extend from the incision channel 120 to expose the first blade cutting surface 305A. The second blade 340 also protrudes from the incision channel 120.

A blade distance is defined between the first blade distal end 1120A of the first blade 305 and the second blade proximal end 1125B of the second blade 340. As such, the blade distance is greater in an instance in which the retractable cutting apparatus 300 is in the retracted position than an instance in which the retractable cutting apparatus 300 is in the deployed position.

The guide member 130 is structured to move a specific distance along the incision channel based on the desired blade height. The blade height is defined by the height of the tip of the first blade 305 from the open channel top of the incision channel 120 (e.g., how much the first blade protrudes from the incision channel 120). The blade height is adjustable. As discussed herein, the guide member may be connected to a lead screw mechanism that is structured to change the distance in which the guide member can move in the direction of the second blade 340, such that the blade height is changed (e.g., the farther the guide member can move in the direction of the second blade, the taller the blade height).

The guide member 130 may be positioned within a lumen of the catheter 129 (e.g., a guidewire lumen). The guide member 130 may extend through the given lumen of the catheter and extend into the controller of the catheter as discussed herein. The controller may include a guide member movement actuator that is attached to the guide member. The guide member movement actuator is manipulated to move the guide member.

FIGS. 10 and 11 illustrate an example retractable cutting apparatus 300 in a deployed position. Upon moving the retractable cutting apparatus 300 into the deployed position, the catheter is moveable along the pericardium cavity to allow an incision to be made. The incision may define an incision length that is defined as the length in which the catheter is moved in an instance in which the retractable cutting apparatus 300 is in the deployed position. The incision may be linear due to the movement of the catheter. The retractable cutting apparatus 300 remains generally stationary relative to the catheter while the catheter is moving. As such, the angle between the first cutting apparatus link 310 and the second cutting apparatus link 315 remains constant during the incision (e.g., the lead screw maintains the retractable cutting apparatus in the deployed position until the actuating knob 700 is manipulated).

As the guide member 130 moves in the direction opposite of the second blade 340, the retractable cutting apparatus 300 begins to move to the retracted position. As the guide member 130 moves in the direction opposite of the second blade 340, the connection between the first blade 305 and the second blade 340 moves downward from the incision channel (e.g., in the direction of a bottom of the incision channel 120). The first blade 305 and the second blade 340 may move into the incision channel 120, such that neither the first blade 305 nor the second blade 340 protrude from the incision channel 120. Upon retraction, a sheath (as discussed herein) may be moved to cover the incision channel for removal from the human body.

In one example, a puncture to deliver a guidewire into the pericardial space is performed through heart tissue. When a transvascular approach through the RAA, IVC, or SVC is employed, a closure device may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

In one example, to provide orientational stability of the cutting surface to that of the parietal layer, an OTW introduction is employed for any of the previously disclosed devices, for example, whether through a dedicated lumen in multi-lumen catheter cross-section or ‘Rapid Exchange’ style catheter, or off-center attached cannula, or deflect-resistant catheter. In one example, the delivering catheter comprises radiopaque material randomly distributed or arranged in a pattern for visualization using conventional visualization techniques during use.

Current ECHO/fluoroscopy may not provide the required visualization for certain access applications of the presently disclosed transection devices, for example, gaining guidewire access to the pericardial cavity consistently and repeatedly may be desired. Thus, in one example, the multi-lumen catheter 129 coupled to the presently disclosed transection devices comprises direct visualization allowing the user to watch real-time the advancement of any of the presently disclosed transection devices traverse through various tissue layers until the desired location is reached. Changes in tissue layers that may not be visible under ECHO/fluoroscopy may be easily distinguishable under direct visualization such as tissue/bloodstream (vessel access), myocardium/pericardium (pericardial cavity access), myocardium/pericardium (outside pericardium), among other anatomical features.

Thus, in one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

In one example, a puncture to deliver a guidewire into the pericardial cavity 54 is performed through heart tissue in a transvascular approach. When a transvascular approach through the RAA, IVC, or SVC is employed, a closure device (e.g., occluder) may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

The following exemplary occlusion descriptions relate to a transvascular approach through the RAA, IVC, SVC, or CS using one of the aforementioned transection devices (e.g., incision device 140). In one example, a puncturing tip 115 delivers a wire into the pericardial space through heart tissue. A closure or occlusion device is introduced for hemostasis during the procedure. The closure or occlusion device in one example includes outward or radially directed splines deployed in an expanded configuration. When the guide catheter is removed, the splines or radial members of the closure device contract inwardly towards the unstressed state of the transection device in order to close and seal the opening. The closure device is designed such that a pericardial cutting device can pass through and into the pericardial space.

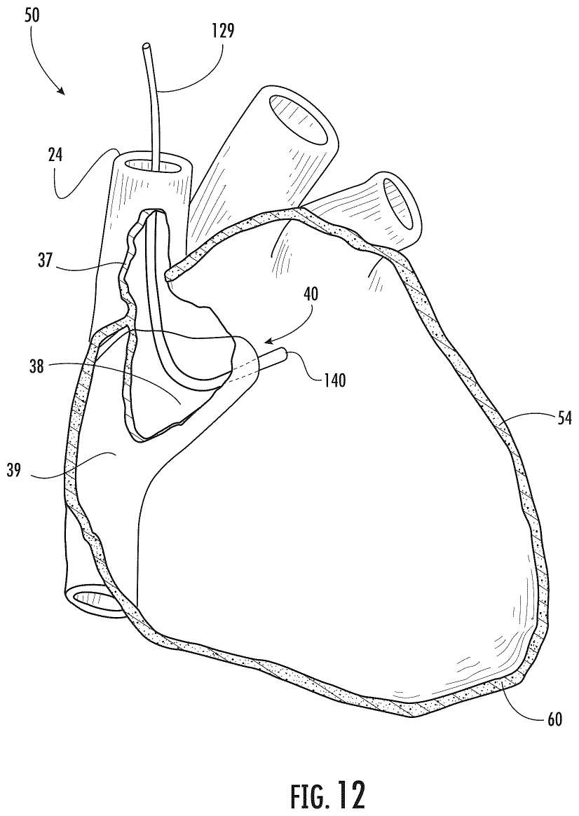

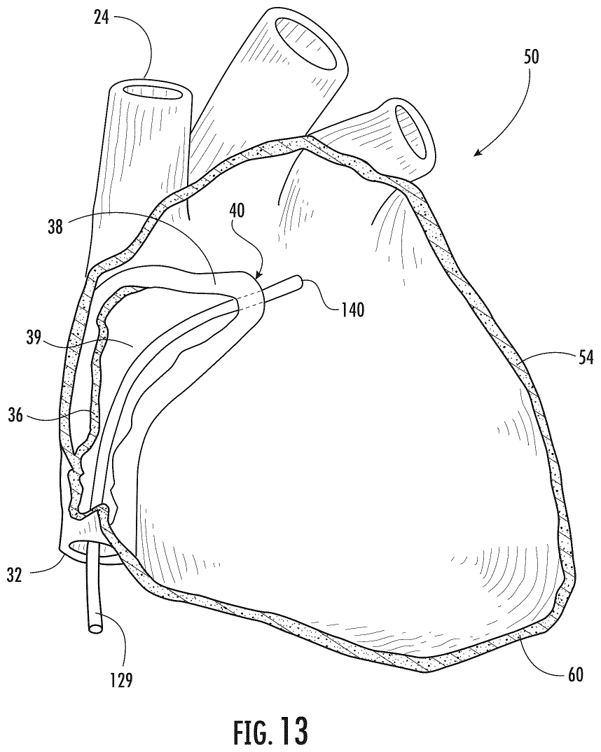

FIGS. 12 and 13 shows exemplary intravascular approaches for delivering the transection devices of the present disclosure to the pericardial cavity 54. Thus, FIG. 12 depicts heart 50 viewed in isolation from the body, with the pericardium 60 or pericardial sac encasing the cardiac muscle (i.e., epicardium, myocardium, and endocardium). The small space which is present between the heart muscle and pericardium 60 represents the pericardial cavity 54.

The presently disclosed transection devices that can be presented to the pericardial cavity 54. In one example via the right atrial appendage 38 (RAA), which is a suitable site for entry into the pericardial cavity 54, is used. Right atrial appendage 38 lies tangential to and between pericardium 60 and the epicardium/epicardial adipose tissue 57. In one example, any of the presently disclosed devices is guided into right atrial appendage 38 via right atrium 39 so as to be positioned substantially in parallel with the wall of pericardium 60 such that when the wall of right atrial appendage 38 is pierced by any of the presently disclosed devices substantially without risk of damaging the epicardium or other heart tissue. Other access routes to the pericardial cavity can be used, for example, direct “puncture out” of SVC or IVC/coronary sinus (CS) and a “puncture into” the pericardium.

In some examples, right atrial appendage 38 is accessed via conventional vena cava routes. FIG. 12 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

FIG. 13 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at apex 40.

Thus, by way of example, a method of reducing pericardial restraint of a subject in need thereof using any of the presently disclosed devices is provided by the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other transvascular-right heart routes to the pericardial cavity 54 are envisaged. Furthermore, left atrial appendage, coronary sinus, and right ventricle pathways are envisaged for transvascular access to the pericardial cavity 54.

Note that the wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

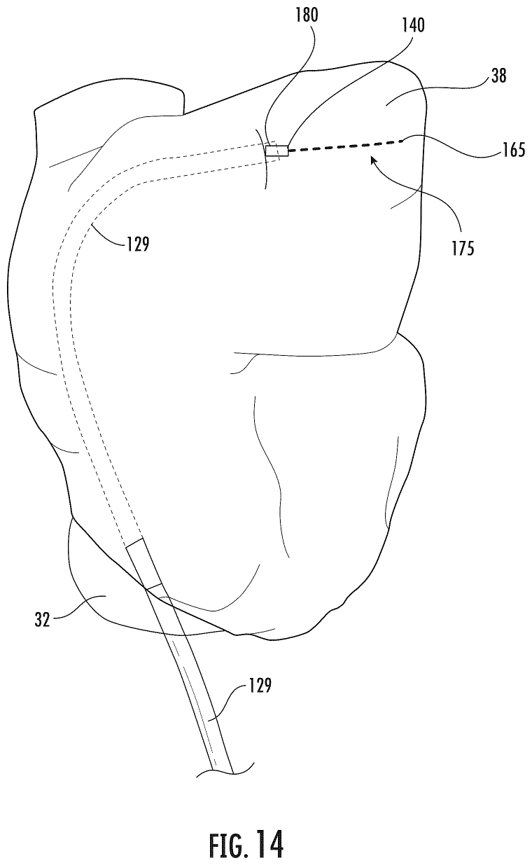

As shown in FIG. 14, any of the presently disclosed devices can be used to create a cut path of a length in a pericardium, e.g., in a parietal layer 55. Thus, a catheter 129, e.g., a steerable catheter can be employed, extending through the IVC, through the RA, and into the RAA, for example, and then into the pericardial cavity 54. The catheter 129 may have one or more steerable segments guiding any of the presently disclosed devices, with a radius of curvature of between about 1 inch and about 5 inches, with an arc length of between about 90° and about 180°. As exemplary shown in FIG. 14, any of the presently disclosed devices can be positioned in the pericardial cavity 54 and can begin a cut path 175 at a start point 165 and ends at endpoint 180 of a length. At least a portion of the parietal layer 55 of the pericardium, and the fibrous pericardium 56, and pericardial adipose tissue 59 are separated along cut path 175. The one or more incisions along the lengths, in a heart with a dysfunction treatable with the present method, cause the pericardium to separate radially about the cut line of the incision path 175, without the removal of pericardial tissue and with a reduction in pericardial restraint. One or more cut paths 175 can be made, and different cut paths, of various lengths can be used to reduce pericardial restraint. In one example, the cut path 175 and its length is pre-operatively determined. Other cut paths and lengths can be used.

In one example, the presently disclosed device further comprises at least one nerve detection device. In one example, the at least one nerve detection device is located on the flexible catheter 129. In one example, the at least one nerve detection device is located adjacent the incision device. In one example, the at least one nerve detection device is located on the puncturing tip/introducer 115. In one example, the at least one nerve detection device is located on the cutting surface.

Any one of the presently disclosed devices can further comprises at least one nerve stimulation device. In one example, the at least one nerve stimulation device is located on the flexible catheter 129. In one example, the at least one nerve stimulation device is located adjacent the incision device. In one example, the at least one nerve stimulation device is located on puncturing tip/introducer 115. In one example, the at least one nerve stimulation device is located on the cutting surface.

In one example, the presently disclosed devices discussed above further comprise an optical channel in the catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the catheter to accommodate a lens coupled to a fiber optic cable, optionally with a light source, e.g., an LED.

A kit, comprising any one of the presently disclosed medical devices, a sheath, a guidewire, and a puncturing tip is provided.

While certain embodiments of the present disclosure have been illustrated with reference to specific combinations of elements, various other combinations may also be provided without departing from the teachings of the present disclosure. Thus, the present disclosure should not be construed as being limited to the particular exemplary embodiments described herein and illustrated in the Figures but may also encompass combinations of elements of the various illustrated embodiments and aspects thereof.

We claim:

1. A medical device, the device comprising:
an incision device structured to be operably coupled to a distal end of a catheter, wherein the incision device comprises an incision channel defined along the incision device, wherein the incision channel defines an open channel top and a channel bottom; and
a retractable cutting apparatus disposed within the incision channel, wherein the retractable cutting apparatus comprises:
a first cutting apparatus link defining a first link distal end and a first link proximal end, wherein the first link distal end is rotatably fixed proximate to a distal end of the incision channel;
a second cutting apparatus link defining a second link distal end and a second link proximal end, wherein the second link distal end is attached to the first link proximal end of the first cutting apparatus link; and
a guide member coupled to the second link proximal end, wherein the guide member is structured to move the first cutting apparatus link and the second cutting apparatus link and cause the retractable cutting apparatus to move between a retracted position and a deployed position,
wherein the first cutting apparatus comprises a first cutting surface proximate to the first link proximal end of the first cutting apparatus link, wherein the first cutting surface is structured to protrude from the incision channel in an instance in which the retractable cutting apparatus is in the deployed position,
wherein the second cutting apparatus link comprises a second cutting surface,
wherein in an instance in which the guide member is moved in a first direction of the first cutting apparatus link and the second cutting apparatus link, the first link proximal end of the first cutting apparatus link and the second link distal end of the second cutting apparatus link are structured to move in a direction of the open channel top to the deployed position, and
wherein in an instance in which the guide member is moved in a second direction opposite of the first cutting apparatus link and the second cutting apparatus link, the first link proximal end of the first cutting apparatus link and the second link distal end of the second cutting apparatus link are structured to move in a direction of the channel bottom to the retracted position.

2. The medical device of claim 1, wherein the second cutting apparatus link defines a first blade receiving aperture that is structured to receive the first cutting surface in an instance in which the retractable cutting apparatus is in the retracted position.

3. The medical device of claim 1, wherein upon movement to the deployed position, the first cutting surface is structured to make an incision along a pericardium upon movement of the catheter.

4. The medical device of claim 1, wherein at least one of the first cutting surface and the second cutting surface comprise at least one of a blade or an electrode.

5. The medical device of claim 1, wherein the incision channel comprises a channel protrusion structured to prevent the first cutting surface and a second cutting surface from being parallel along the incision channel.

6. The medical device of claim 1, wherein the channel protrusion causes an angle between the first cutting surface and a secondary cutting surface to be less than 180 degrees to assist the movement between the retracted position and the deployed position.

7. The medical device of claim 1, wherein the first cutting surface remains stationary along the incision channel in an instance in which the catheter is being moved.

8. The medical device of claim 1, wherein the first cutting surface is located on a bottom surface of the first cutting apparatus link and the second cutting surface is located on a top surface of the second cutting apparatus link.

9. The medical device of claim 8, wherein the first and second cutting surfaces are angled relative to each other to create opposing cutting surfaces.

10. The medical device of claim 1, wherein at least one of the first cutting surface and the second cutting surface comprises an electrode.

11. The medical device of claim 1, wherein the first cutting surface comprises an electrode and the second cutting surface comprises a mechanical blade.

12. The medical device of claim 1, wherein the first cutting surface is a blade rigidly attached to the first cutting apparatus link and the second cutting surface is a blade rigidly attached to the second cutting apparatus link.

13. The medical device of claim 1, wherein the first cutting surface defines a blade height that is defined by the amount the first cutting surface protrudes from the incision channel in the deployed position, wherein the blade height is adjustable.

14. The medical device of claim 1, wherein the first cutting apparatus link further comprises a puncturing tip adjacent to the first link proximal end.

15. The medical device of claim 1, further comprising a guide member movement actuator attached to the guide member, wherein the guide member movement actuator is manipulated to move the guide member.

16. A medical device, the device comprising:
an incision device structured to be operably coupled to a distal end of a catheter, wherein the incision device comprises an incision channel defined along the incision device, wherein the incision channel defines an open channel top and a channel bottom; and
a retractable cutting apparatus disposed within the incision channel, wherein the retractable cutting apparatus comprises:
a first cutting apparatus link defining a first link distal end and a first link proximal end, wherein the first link distal end is rotatably fixed proximate to a distal end of the incision channel;
a second cutting apparatus link defining a second link distal end and a second link proximal end, wherein the second link distal end is attached to the first link proximal end of the first cutting apparatus link; and
a guide member coupled to the second link proximal end, wherein the guide member is structured to move the first cutting apparatus link and the second cutting apparatus link and cause the retractable cutting apparatus to move between a retracted position and a deployed position,
wherein the first cutting apparatus comprises a first cutting surface proximate to the first link proximal end of the first cutting apparatus link, wherein the first cutting surface is structured to protrude from the incision channel in an instance in which the retractable cutting apparatus is in the deployed position,
wherein the second cutting apparatus link comprises a second cutting surface,
wherein in an instance in which the guide member is moved in a first direction of the first cutting apparatus link and the second cutting apparatus link, the first link proximal end of the first cutting apparatus link and the second link distal end of the second cutting apparatus link are structured to move in a direction of the open channel top to the deployed position, and
wherein the first and second cutting surfaces are positioned in an angled configuration relative to each other to thereby create opposing cutting surfaces.

17. The medical device of claim 16, wherein the first cutting apparatus link further comprises a puncturing tip adjacent to the first link proximal end.

18. The medical device of claim 16, wherein the first cutting apparatus link further comprises a puncturing tip adjacent to the first link proximal end, and wherein the first cutting surface comprises an electrode and the second cutting surface comprises a mechanical blade.

19. The medical device of claim 16, wherein the incision channel comprises a channel protrusion structured to prevent the first cutting surface and a second cutting surface from being parallel along the incision channel.

20. The medical device of claim 16, wherein the first cutting surface is a blade rigidly attached to the first cutting apparatus link and the second cutting surface is a blade rigidly attached to the second cutting apparatus link.
